(19) United States
(12) Patent Application Publication
(10) Pub. No.: US 2023/0270382 A1
(43) Pub. Date: Aug. 31, 2023

(54) WEARABLE ACTIVITY TRACKING AND BIOMETRIC MONITORING DEVICE

(71) Applicant: Alec Linarte, Tampa, FL (US)

(72) Inventor: Alec Linarte, Tampa, FL (US)

(21) Appl. No.: 18/168,718

(22) Filed: Feb. 14, 2023

Related U.S. Application Data
(60) Provisional application No. 63/313,764, filed on Feb. 25, 2022.

(51) Int. Cl. A61B 5/00

(52) U.S. Cl. CPC A61B 5/681

(57) ABSTRACT

A discrete biometric monitoring device may be constructed and arranged to be affixed directly to traditional wristwear such that traditional wristwear may be transformed into a smart device without altering the appearance of the traditional wristwear. The stand-alone biometric monitoring device may include activity tracking sensors including pedometer functionality, accelerometers, gyroscopes, thermometers, altimeters, heart rate sensors, or the like to calculate user steps, body temperature, active time, VO2 max, altitude, speed, heart rate, heart rate variability, and the like. The device may be in operable communication with a software application executed on a separate computing device such as a smartphone such that a user may receive and review biometric data measured, stored, and communicated by the device.

### CROSS-REFERENCE TO RELATED APPLICATIONS

This application claims the benefit of U.S. provisional patent application No. 63/313,764 filed on Feb. 25, 2022.

### TECHNICAL FIELD

The embodiments generally relate to the field of wearable biometric monitoring devices.

### BACKGROUND

Wearable activity tracking and biometric monitoring devices may take the form of chest straps or watches which serve to replace traditional wearables or wristwear, such as conventional watches, in order to provide useful biometric data related to the wearer. However, these activity trackers lack the structure and function to retrofit existing wearables from conventional devices to more modern, smart activity tracking devices.

### SUMMARY

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

A stand-alone activity tracking and biometric monitoring device may be constructed and arranged to be affixed directly to traditional wristwear lacking biometric monitoring functionality.

The stand-alone activity tracking and biometric monitoring device may provide for activity tracking such as, but not limited to heart rate monitoring, sleep tracking, steps taken, or the like.

The stand-alone activity tracking and biometric monitoring device may be in operable communication with a software application executed on a computing device such as a smartphone such that a user may receive and review biometric data.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a perspective view of a biometric monitoring device as described herein;

FIG. 2 illustrates a side view of a biometric monitoring device as described herein;

FIG. 3 illustrates a side view of a biometric monitoring device as described herein;

FIG. 4 illustrates a top view of a biometric monitoring device as described herein;

FIG. 5 illustrates a bottom view of a biometric monitoring device as described herein;

FIG. 6 illustrates a partial cross-sectional view of a biometric monitoring device as described herein; and

FIG. 7 illustrates a side view of a biometric monitoring device attached to a wearable item, as described herein;

The drawings are not necessarily to scale, and certain features and certain views of the drawings may be shown exaggerated in scale or in schematic in the interest of clarity and conciseness.

### DETAILED DESCRIPTION

The specific details of the single embodiment or variety of embodiments described herein are to the described system and methods of use. Any specific details of the embodiments are used for demonstration purposes only and no unnecessary limitations or inferences are to be understood from there.

It is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments described herein relate to a discrete biometric monitoring device constructed and arranged to be affixed directly to traditional wristwear. The biometric monitoring device may be affixed to wristwear via a connection means such as an adhesive patch, magnet(s), or the like. According to some embodiments, the discrete biometric monitoring device includes an elastic adhesive patch constructed and arranged to fix the device to an conventional wristwatch.

The stand-alone biometric monitoring device may include a sensor suite including activity tracking sensors or biometric sensors configured to read biometric devices or calculating physiological or behavioral biometric inputs including pedometer functionality, accelerometers, gyroscopes, thermometers, altimeters, heart rate sensors, or the like to calculate user steps, body temperature, active time, VO2 max, altitude, speed, heart rate, heart rate variability, stored glycogen and glycogen depletion, and others. Biometric sensors may include at least one transducer configured to change biometric inputs into electrical signals which may be communicated to a processor or the like within the device. According to some embodiments, any of the activity tracking sensors may be exposed to a user wearing the device and, in some cases, may be in contact with a wearer during use.

The stand-alone biometric monitoring device may include at least one processor, at least one memory, and at least one control logic in operable communication with any of the pedometer functionality, accelerometers, thermometers, altimeters, or heart rate sensors. Additionally, the at least one processor, at least one memory, and at least one control logic may be in operable communication with data storage devices, Bluetooth modules, wireless internet connection modules, or near-field communication modules. The at least one control logic may be configured to receive data from any of the pedometer, accelerometers, thermometers, altimeters, or heart rate sensors. The stand-alone biometric monitoring device and control logic may be in operable communication with a software application executed on a separate computing device such as a smartphone such that a user may receive and review biometric data measured, stored, and communicated by the device.

The stand-alone biometric monitoring device may include at least one processor, a memory, input/output devices, and a network interface constructed and arranged to facilitate communication with a network. The memory may include computer-readable application instructions i.e. control logic, configured to implement certain embodiments described herein, and a database, comprising various data accessible by the application instructions. In some embodiments, the steps and actions of the application instructions described herein are embodied directly in the hardware, in a software program product executed by a processor, or in a combination of the two. A software program product may reside in RAM, flash memory, ROM memory, EPROM memory, EEPROM memory, registers, a hard disk, a removable disk, a CD-ROM, or any other form of storage medium known in the art. An exemplary storage medium may be coupled to the processor such that the processor can read information from, and write information to, the storage medium. In the alternative, the storage medium may be integrated into the processor. Further, in some embodiments, the processor and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions can execute entirely on the devices' computer, partly on the devices' computer, as a stand-alone software package, or partly on the devices' computer and partly on a remote computer. In the latter scenario, the remote computer can be connected to the devices' computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, to perform aspects of the present disclosure.

In some embodiments, the application instructions can be downloaded to a computing/processing device from a computer readable storage medium, or to an external computer or external storage device via a network. A network adapter card or network interface in each computing/processing device may receive computer readable program instructions from the network and forwards the computer readable application instructions for storage in a computer readable storage medium within the respective computing or processing device.

In some embodiments, the computer system includes a network interface to communicate with a network. In some embodiments, the network interface is configured to allow data to be exchanged between the computer system and other devices attached to the network, such as other computer systems, or between nodes of the computer system. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network or protocol.

The network may be configured to facilitate operable communication between the computer system and any of administrator devices, remote servers or remote computer systems, or cloud network integration.

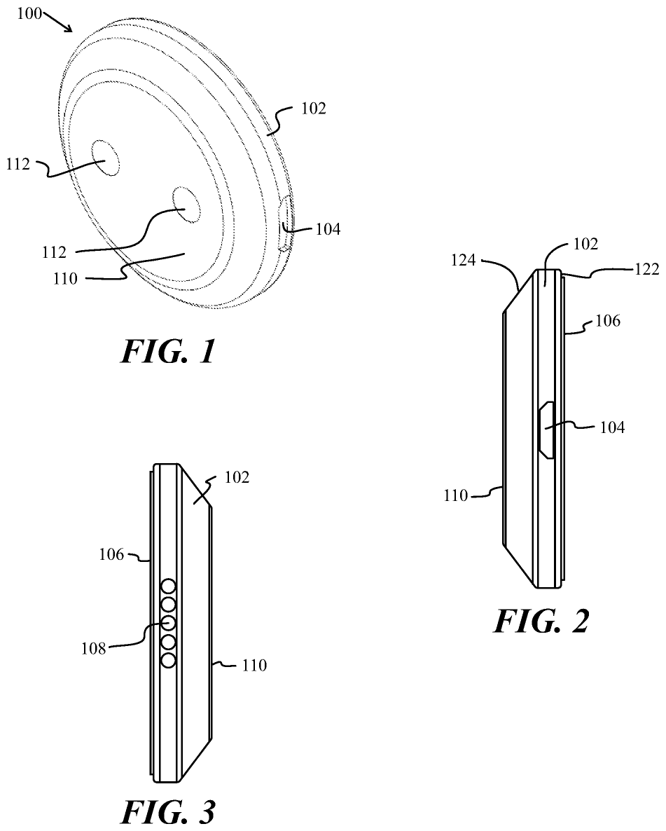

FIG. 1 illustrates a perspective view of a biometric monitoring device 100 including a housing 102 having a user facing surface 110 having at least one user facing sensor 112. The housing 102 may include flexible, comfortable, and skin-safe materials such as silicone. According to some embodiments, the housing 102 may be approximately 32 millimeters in diameter or length and approximately 6 millimeters thick. The device 110 may include at least one port 104 constructed and arranged for charging of a battery within the device 100 or the transfer of data from the device 100 to an external device, such as a computer. The housing, and device in general, may be generally disc shaped, puck shaped, truncated-cone shaped, dome shaped, hemisphere shaped, or the like. According to other embodiments, the housing, and device in general, may be approximately triangular, rectangular, square, or oval in shape and dimension. The housing may be generally configured to be discretely attached to the underside of traditional, non-processor-integrated wristwear such that the device is partially or substantially unnoticeable when in use. The housing may be constructed and arranged to seat beneath wristwear, such as a conventional wristwatch, while remaining in contact with a wearer's skin and wrist while allowing the conventional wristwatch to remain visible and unimpacted by the presence of the device.

FIG. 2 illustrates a side view of a biometric monitoring device including a housing 102 having a user facing surface 110 opposite a wristwear facing surface 122. A connection substrate 106 may be disposed on the wristwear facing surface 122 such that the connection substrate may be affixed to a wristwear device such as a watch, bracelet, bangle, or the like. The connection substrate may be a magnet, magnetic substrate, or an adhesive pad configured to attach the device to conventional devices. The housing 102 may define a chamfered circumferential face 124 constructed and arranged to provide a comfortable wearing experience to a user such that no uncomfortable edges or seams come into contact with a wearers skin during use.

FIG. 3 illustrates a side view of a biometric monitoring device including a housing 102 having a user facing surface 110 having a connection substrate 106 disposed thereon. According to some embodiments, the connection substrate 106 may be integrated with the housing 102 such that the connection substrate 106 is flush with the wristwear facing surface 122. According to some embodiments, the connection substrate 106 may include an adhesive, elastic or pliable material configured to elastically conform to a wristwear device. At least one indicator light 108 may be integrated with the housing 102 and in some embodiments may include a plurality of indicator lights 108 configured to convey information such as battery charge, device on or off modes, error modes, or the like.

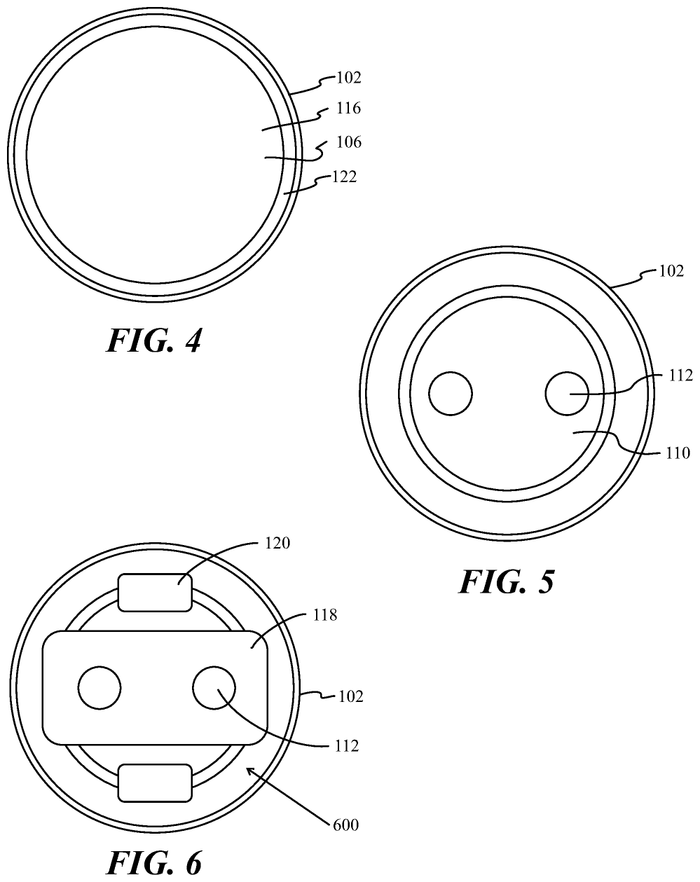

FIG. 4 illustrates a top view of a biometric monitoring device having a housing 102 including a connection substrate 106 disposed on or integrated with a wristwear facing surface 122. An adhesive layer 116 may be disposed on the connection substrate 106 such that the connection substrate may be affixed to a wristwear device such as a watch, bracelet, bangle, or the like.

FIG. 5 illustrates a bottom view of a biometric monitoring device having a housing 102 including a user facing surface 110 opposite the wristwear facing surface 122 as depicted in FIG. 4. The device may include at least one user facing sensor 112 such as, but not limited to, pedometers, accelerometers, thermometers, altimeters, or heart rate sensors.

FIG. 6 illustrates a partial cross-sectional view of a biometric monitoring device including a housing 102 constructed and arranged to house a sensor suite 600 including at least one user facing sensor 112, at least one battery 120, and components 118 such as at least one processor, at least one memory, at least one data storage device, Bluetooth modules, wireless internet connection modules, near-field communication modules, or the like. The sensor suite 600 may include activity tracking sensors or biometric sensors configured to read biometric devices or calculating physiological or behavioral biometric inputs including pedometer functionality, accelerometers, gyroscopes, thermometers, altimeters, heart rate sensors, or the like to calculate user steps, body temperature, active time, VO2 max, altitude, speed, heart rate, heart rate variability, stored glycogen and glycogen depletion, and others.

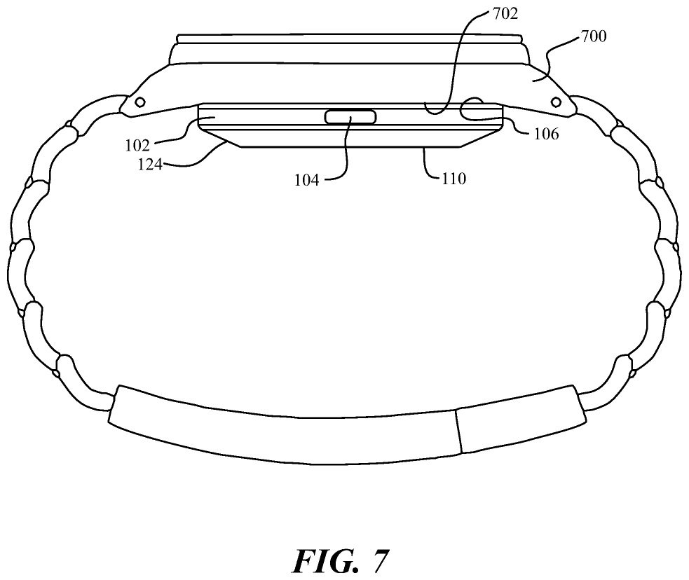

FIG. 7 illustrates a side view of a biometric monitoring device attached to a conventional item 700, for example, an analog watch. The item 700 may have a device mounting surface 702 onto which the biometric monitoring device may be attached. The biometric monitoring device may include a housing 102 having a user facing surface 110 opposite a wristwear facing surface 122. A connection substrate 106 may be disposed on the wristwear facing surface 122 such that the connection substrate may be affixed to the device mounting surface 702 of the item 700, such as a watch, bracelet, bangle, or the like. The connection substrate may be a magnet, magnetic substrate, or an adhesive pad configured to attach the device to conventional devices. The housing 102 may define a chamfered circumferential face 124 constructed and arranged to provide a comfortable wearing experience to a user such that no uncomfortable edges or seams meet a wearers skin during use.

One of ordinary skill in the art will understand and appreciate that the depicted variations within FIG. 1 through FIG. 7 are exemplary only and the shape, arrangement, and configuration of the housing, ports, sensors, indicators, and connection substrate may take many forms while still falling within the scope of this disclosure. As non-limiting examples, the housing may be generally square, rectangular, triangular, or the like. Similarly, ports, sensors, indicators, and the like may take a variety of sizes, shapes, configurations, and locations within or on the device. Similarly, the connection substrate may include a plurality of connection substrates affixed to the housing such as a plurality of magnets or plurality of adhesive substrates.

The following description of variants is only illustrative of components, elements, acts, products, and methods considered to be within the scope of the invention and are not in any way intended to limit such scope by what is specifically disclosed or not expressly set forth. The components, elements, acts, products, and methods as described herein may be combined and rearranged other than as expressly described herein and are still considered to be within the scope of the invention.

According to variation 1, a biometric monitoring device may include a housing may include a user facing surface opposite a device facing surface, the housing defining at least one throughhole in the user facing surface; at least one biometric sensor partially disposed within the housing and partially disposed within the at least one throughhole such that the at least one biometric sensor; a sensor suite at least partially disposed within the housing; and at least one connection substrate disposed on the device facing surface, the at least one connection substrate being configured to facilitate attachment of the biometric monitoring device to a device.

Variation 2 may include a biometric monitoring device as in variation 1, wherein the sensor suite comprises at least one of a pedometer, accelerometer, gyroscope, thermometer, altimeter, or heart rate sensor.

Variation 3 may include a biometric monitoring device as in variation 1 or 2, wherein the sensor suite is configured to determine at least one of user steps, body temperature, active time, VO2 max, altitude, speed, heart rate, or heart rate variability.

Variation 4 may include a biometric monitoring device as in any of variations 1 through 3, wherein the housing is at least one of generally disc shaped, generally puck shaped, generally truncated-cone shaped, generally dome shaped, or generally hemisphere shaped.

Variation 5 may include a biometric monitoring device as in any of variations 1 through 4, wherein the housing comprises silicone.

Variation 6 may include a biometric monitoring device as in any of variations 1 through 5, wherein the housing is generally disk shaped and the housing is approximately 32 millimeters in diameter and approximately 6 millimeters thick.

Variation 7 may include a biometric monitoring device as in any of variations 1 through 6, wherein the housing is constructed and arranged to attach to a wristwear while being in contact with a wearer's skin while allowing the wristwear to remain visible and unimpacted by the presence of the biometric monitoring device.

Variation 8 may include a biometric monitoring device as in any of variations 1 through 7, wherein the housing is configured to be discretely attached to a non-processor-integrated wristwear such that the biometric monitoring device is approximately unnoticeable when in use.

Variation 9 may include a biometric monitoring device as in any of variations 1 through 8 and may further include at least one communication protocol port constructed and arranged for at least one of charging of a battery within the device or the transfer of data from the device to an external device.

Variation 10 may include a biometric monitoring device as in any of variations 1 through 9, wherein the connection substrate comprises at least one of a magnet, a magnetic substrate, an adhesive, or an adhesive pad.

Variation 11 may include a biometric monitoring device as in any of variations 1 through 10, wherein the housing defines a chamfered circumferential face constructed and arranged to provide a comfortable wearing experience to a user.

Variation 12 may include a biometric monitoring device as in any of variations 1 through 11, wherein the at least one connection substrate is integrated with the housing.

According to variation 13, a biometric monitoring device may include a generally disc-shaped housing may include a user facing surface opposite a device facing surface, the housing defining at least one throughhole in the user facing surface; at least one biometric sensor partially disposed within the housing and partially disposed within the at least one throughhole such that the at least one biometric sensor; a sensor suite at least partially disposed within the housing, the sensor suite may include at least one of a pedometer, accelerometer, gyroscope, thermometer, altimeter, or heart rate sensor, wherein the sensor suite is configured to determine at least one of user steps, body temperature, active time, VO2 max, altitude, speed, heart rate, or heart rate variability; and at least one connection substrate disposed on the device facing surface, the at least one connection substrate being configured to facilitate attachment of the biometric monitoring device to a device.

Variation 14 may include a biometric monitoring device as in variation 13, wherein the generally disc-shaped housing comprises silicone.

Variation 15 may include a biometric monitoring device as in any of variations 13 through 14, wherein the generally disc-shaped housing is approximately 32 millimeters in diameter and approximately 6 millimeters thick.

Variation 16 may include a biometric monitoring device as in any of variations 13 through 15, wherein the generally disc-shaped housing is constructed and arranged to attach to a wristwear while being in contact with a wearer's skin while allowing the wristwear to remain visible and unimpacted by the presence of the biometric monitoring device.

Variation 17 may include a biometric monitoring device as in any of variations 13 through 16, wherein the generally disc-shaped housing is configured to be discretely attached to a non-processor-integrated wristwear such that the biometric monitoring device is approximately unnoticeable when in use.

Variation 18 may include a biometric monitoring device as in any of variations 13 through 17, further including at least one communication protocol port constructed and arranged for at least one of charging of a battery within the device or the transfer of data from the device to an external device.

Variation 19 may include a biometric monitoring device as in any of variations 12 through 18, wherein the connection substrate comprises at least one of a magnet, a magnetic substrate, an adhesive, or an adhesive pad.

According to variation 20, a biometric monitoring device may include a generally disc-shaped housing may include a user facing surface opposite a device facing surface, the housing defining at least one throughhole in the user facing surface, wherein the generally disc-shaped housing is approximately 32 millimeters in diameter and approximately 6 millimeters thick; at least one biometric sensor partially disposed within the housing and partially disposed within the at least one throughhole such that the at least one biometric sensor; a sensor suite at least partially disposed within the housing, the sensor suite may include at least one of a pedometer, accelerometer, gyroscope, thermometer, altimeter, or heart rate sensor, wherein the sensor suite is configured to determine at least one of user steps, body temperature, active time, VO2 max, altitude, speed, heart rate, or heart rate variability; at least one communication protocol port constructed and arranged for at least one of charging of a battery within the device or the transfer of data from the device to an external device; and at least one connection substrate disposed on the device facing surface, wherein the connection substrate comprises at least one of a magnet, a magnetic substrate, an adhesive, or an adhesive pad, and wherein the at least one connection substrate being configured to facilitate attachment of the biometric monitoring device to a device.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for anyone of the elements in the claims below or that a single element can be substituted for two or more elements in a claim. Although elements can be described above as acting in certain combinations, and even initially claimed as such, it is to be expressly understood that one or more elements from a claimed combination can, in some cases, be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible considering the above teachings without departing from the following claims.

I/We claim:

1. A biometric monitoring device comprising:
a housing comprising a user facing surface opposite a device facing surface, the housing defining at least one throughhole in the user facing surface;
at least one biometric sensor partially disposed within the housing and partially disposed within the at least one throughhole such that the at least one biometric sensor;
a sensor suite at least partially disposed within the housing; and
at least one connection substrate disposed on the device facing surface, the at least one connection substrate being configured to facilitate attachment of the biometric monitoring device to a device.

2. The biometric monitoring device of claim 1, wherein the sensor suite comprises at least one of a pedometer, accelerometer, gyroscope, thermometer, altimeter, or heart rate sensor.

3. The biometric monitoring device of claim 1, wherein the sensor suite is configured to determine at least one of user steps, body temperature, active time, VO2 max, altitude, speed, heart rate, or heart rate variability.

4. The biometric monitoring device of claim 1, wherein the housing is at least one of generally disc shaped, generally puck shaped, generally truncated cone shaped, generally dome shaped, or generally hemisphere shaped.

5. The biometric monitoring device of claim 1, wherein the housing comprises silicone.

6. The biometric monitoring device of claim 1, wherein the housing is generally disk shaped and the housing is approximately 32 millimeters in diameter and approximately 6 millimeters thick.

7. The biometric monitoring device of claim 1, wherein the housing is constructed and arranged to attach to a wristwear while being in contact with a wearer's skin while allowing the wristwear to remain visible and unimpacted by the presence of the biometric monitoring device.

8. The biometric monitoring device of claim 1, wherein the housing is configured to be discretely attached to a non-processor-integrated wristwear such that the biometric monitoring device is approximately unnoticeable when in use.

9. The biometric monitoring device of claim 1, further comprising:
at least one communication protocol port constructed and arranged for at least one of charging of a battery within the device or the transfer of data from the device to an external device.

10. The biometric monitoring device of claim 1, wherein the connection substrate comprises at least one of a magnet, a magnetic substrate, an adhesive, or an adhesive pad.

11. The biometric monitoring device of claim 1, wherein the housing defines a chamfered circumferential face constructed and arranged to provide a comfortable wearing experience to a user.

12. The biometric monitoring device of claim 1, wherein the at least one connection substrate is integrated with the housing.

13. A biometric monitoring device comprising:
a generally disc-shaped housing comprising a user facing surface opposite a device facing surface, the housing defining at least one throughhole in the user facing surface;
at least one biometric sensor partially disposed within the housing and partially disposed within the at least one throughhole such that the at least one biometric sensor;
a sensor suite at least partially disposed within the housing, the sensor suite comprising at least one of a pedometer, accelerometer, gyroscope, thermometer, altimeter, or heart rate sensor, wherein the sensor suite is configured to determine at least one of user steps, body temperature, active time, VO2 max, altitude, speed, heart rate, or heart rate variability; and
at least one connection substrate disposed on the device facing surface, the at least one connection substrate being configured to facilitate attachment of the biometric monitoring device to a device.

14. The biometric monitoring device of claim 1, wherein the generally disc-shaped housing comprises silicone.

15. The biometric monitoring device of claim 1, wherein the generally disc-shaped housing is approximately 32 millimeters in diameter and approximately 6 millimeters thick.

16. The biometric monitoring device of claim 1, wherein the generally disc-shaped housing is constructed and arranged to attach to a wristwear while being in contact with a wearer's skin while allowing the wristwear to remain visible and unimpacted by the presence of the biometric monitoring device.

17. The biometric monitoring device of claim 1, wherein the generally disc-shaped housing is configured to be discretely attached to a non-processor-integrated wristwear such that the biometric monitoring device is approximately unnoticeable when in use.

18. The biometric monitoring device of claim 1, further comprising:
at least one communication protocol port constructed and arranged for at least one of charging of a battery within the device or the transfer of data from the device to an external device.

19. The biometric monitoring device of claim 1, wherein the connection substrate comprises at least one of a magnet, a magnetic substrate, an adhesive, or an adhesive pad.

20. A biometric monitoring device comprising:
a generally disc-shaped housing comprising a user facing surface opposite a device facing surface, the housing defining at least one throughhole in the user facing surface, wherein the generally disc-shaped housing is approximately 32 millimeters in diameter and approximately 6 millimeters thick;
at least one biometric sensor partially disposed within the housing and partially disposed within the at least one throughhole such that the at least one biometric sensor;
a sensor suite at least partially disposed within the housing, the sensor suite comprising at least one of a pedometer, accelerometer, gyroscope, thermometer, altimeter, or heart rate sensor, wherein the sensor suite is configured to determine at least one of user steps, body temperature, active time, VO2 max, altitude, speed, heart rate, or heart rate variability;
at least one communication protocol port constructed and arranged for at least one of charging of a battery within the device or the transfer of data from the device to an external device; and
at least one connection substrate disposed on the device facing surface, wherein the connection substrate comprises at least one of a magnet, a magnetic substrate, an adhesive, or an adhesive pad, and wherein the at least one connection substrate being configured to facilitate attachment of the biometric monitoring device to a device.
